(19) United States
(12) Patent Application Publication
(10) Pub. No.: US 2008/0058926 A1
(43) Pub. Date: Mar. 6, 2008

(54) Percutaneous breast and buttock modification

(76) Inventor: Stephen Solomon, New York, NY (US)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/890,164

(22) Filed: Aug. 3, 2007

Related U.S. Application Data
(63) Continuation-in-part of application No. 11/317,957, filed on Dec. 22, 2005.
(60) Provisional application No. 60/638,565, filed on Dec. 22, 2004.

(51) Int. Cl. A61F 2/52

(52) U.S. Cl. 623/7

(57) ABSTRACT

An apparatus for breast and buttock modification is provided which includes first and second tubes that extend alongside each other, and each of which has a lumen. The apparatus also includes an inflatable balloon having a valve that is detachably coupled to a distal end of the second tube such that a portion of the distal end extends through the valve. The balloon is adapted to be introduced to a space in a body and inflated in the space in the body by fluid introduced through the valve through the second tube. And the second tube is detached from the inflated balloon to leave the inflated balloon positioned in the space in the body.

### CROSS REFERENCE TO RELATED APPLICATION

The present application is a Continuation-in-Part of application Ser. No. 11/317,957, filed Dec. 22, 2005, which is based upon and claims the benefit of priority of U.S. provisional application Ser. No. 60/638,565, filed on Dec. 22, 2004. The entire contents of application Ser. No. 11/317,957 and provisional application Ser. No. 60/638,565 are incorporated herein by reference.

### FIELD OF THE INVENTION

The present invention relates to methods and apparatuses for performing breast and buttock modifications, such as lifts and augmentations, percutaneously.

### BACKGROUND OF THE INVENTION

Historically, breast augmentation is used to enlarge the size of a breast of a patient for cosmetic reasons or to reconstruct or restructure the breast. Typically, breast augmentation is performed surgically from an incision point on the breast. Surgery is performed by inserting an implant through the incision point and placing it under the breast.

Recently, endoscopy is being used for performing augmentation of the breast. An endoscope provides direct visualization of the surgery performed inside the body of the patient. Visualization provides more control in the process of breast and buttock augmentation. However, with the endoscopic guidance, it is difficult to visualize and access the space behind the breast muscle during the breast augmentation. Similarly visualization behind the buttock muscle is invasive using surgical technique.

In light of the above, there is a need of a method and apparatus for performing breast and buttock augmentation percutaneously and reducing the risks and costs associated with surgery during the breast and buttock modification.

### SUMMARY OF THE INVENTION

An object of the present invention is to provide an apparatus and a method to perform breast and buttock augmentation.

Another object of the present invention is to provide an apparatus and a method for breast and buttock lift.

According to an embodiment of the present invention, an apparatus for breast and buttock augmentation includes one or more tubes that extend alongside each other. An inflatable implant having a self sealing valve is detachably coupled to the distal end of one of the tubes such that a portion of the distal end of the tube extends through the self-sealing valve. The apparatus is introduced percutaneously, in the body of a patient through a point on the breast of the patient with the help of image guidance. The implant is adapted to be introduced to a pre-determined space behind the breast in the body of the patient. The implant is inflated in the space by a fluid introduced from the valve through the feeding tube. Further, the tube is detached from the inflated implant to leave the inflated implant positioned in the pre-determined space in the body.

According to another embodiment of the present invention, an apparatus for breast and buttock augmentation is introduced percutaneously or endoscopically from an incision point on the body of a patient. The incision point may be a naval, a pre existing scar or a point under the arm pit. The apparatus includes a tube and an implant. The implant is attached to the tube by a self sealing valve. The implant is inflated after being positioned into a pre-determined space behind the breast of a patient.

According to another embodiment of the present invention, a combined instrument with one or more passages for irrigation and suction are also provided to clean the pre-determined space in case of bleeding.

According to another embodiment of the present invention, the combined instrument includes one or more passages for cutting and cauterization of breast tissues.

According to another embodiment of the present invention, an apparatus for lifting a breast after beast augmentation is provided. The apparatus includes a bone anchor, a tissue anchor, and a suture. The bone anchor is adapted to be screwed into a rib behind the breast. The tissue anchor includes a plurality of barbs to be lodged in the breast tissues. The plurality of barbs are connected to the tissue anchor. The tissue anchor and the bone anchor are connected to each other through the suture. The length of the suture is pre set to lift the breast by a desired amount. The lifting of the breast is performed by adjusting the distance between tissue anchor and the bone anchor. Similarly a buttock lift can be performed anchoring to the pelvic bone.

### BRIEF DESCRIPTION OF DRAWINGS

FIG. 1 illustrates one or more paths marked on the body of a patient, which extends from an incision point to a pre-determined space behind the breasts, in accordance with an embodiment of the present invention;

FIG. 2 illustrates a needle inserted into the breast of the patient, in accordance with an embodiment of the present invention;

FIG. 3 illustrates a cannula inserted into the breast of the patient, in accordance with an embodiment of the present invention;

FIG. 4 illustrates a guide wire inserted through the cannula into the breast of the patient, in accordance with an embodiment of the present invention;

FIG. 5 illustrates a sheath inserted over the guide wire and through the cannula into the breast of the patient, in accordance with an embodiment of the present invention;

FIG. 6 illustrates a detailed view of a catheter, in accordance with an embodiment of the present invention;

FIG. 7 illustrates a cross-sectional view of the catheter, in accordance with an embodiment of the present invention;

FIG. 8 illustrates the catheter inserted over a guide wire through a sheath into the breast of the patient, in accordance with an embodiment of the present invention;

FIG. 9 illustrates the breast of the patient after augmentation, in accordance with an embodiment of the present invention;

FIG. 10 illustrates a partial view of an endotube and an obturator for inserting into the body of the patient, in accordance with an embodiment of the present invention;

FIG. 11 illustrates a schematic view of the endotube that is partially inserted in the body of the patient, in accordance with an embodiment of the present invention;

FIGS. 12 and 13 illustrate an isometric view from two different ends of the combined instrument, in accordance with an embodiment of the present invention;

FIG. 14 illustrates a cross sectional view of the combined instrument, in accordance with an embodiment of the present invention;

FIG. 15 illustrates a sectional view taken on the section lines 5-5 of the combined instrument of FIG. 14 showing the irrigation passage, in accordance with an embodiment of the present invention;

FIG. 16A illustrates a sectional view taken on the section lines 6-6 of the combined instrument of FIG. 14 showing the suction passage, in accordance with an embodiment of the present invention;

FIG. 16B illustrates a detailed view of the adjustable suction tip on the combined instrument, in accordance with an embodiment of the present invention;

FIG. 17A illustrates a sectional view taken on the section lines 7-7 of the combined instrument of FIG. 14 showing the electro-surgical, electro-cauterizing and endoscopic viewing passage of the combined instrument, in accordance with an embodiment of the present invention;

FIG. 17B illustrates a detail view of the electro surgical or electro cauterizing head of the combined instrument, in accordance with an embodiment of the present invention;

FIG. 18 illustrates the feeding tube and the implant inserted beneath the breast, in accordance with an embodiment of the present invention;

FIGS. 19A and 19B illustrate the position of the implant over the feeding tube, in accordance with an embodiment of the present invention;

FIG. 20 illustrates filing of the implant through the feeding tube, in accordance with an embodiment of the present invention;

FIG. 21 illustrates a sectional view of the breast showing the implant in place;

FIG. 22 illustrates a plan view of a dissector used in separating tissues, in accordance with an embodiment of the present invention;

FIG. 23 illustrates a tissue anchor needle inserted through a trocar needle into the breast of the patient, in accordance with an embodiment of the present invention;

FIG. 24 illustrates deployment of barbs inside the breast by the tissue anchor needle, in accordance with an embodiment of the present invention;

FIG. 25 illustrates a bone anchor introduced into the breast to the desired rib behind the breast of the patient, in accordance with an embodiment of the present invention;

FIG. 26 illustrates adjustment of the bone anchor to desired amount to lift the breast of the patient, in accordance with an embodiment of the present invention; and

FIG. 27 illustrates multiple tissue anchor needles or multiple bone anchor needles inserted into the breast of the patient, in accordance with an embodiment of the present invention.

### DETAILED DESCRIPTION

To perform breast augmentation according to various embodiments of the present invention, an implant is inserted percutaneously, over or under the pectoral muscle from an incision point on the body of the patient using image guidance. According to the present invention, after insertion the implant is inflated to augment the breast.

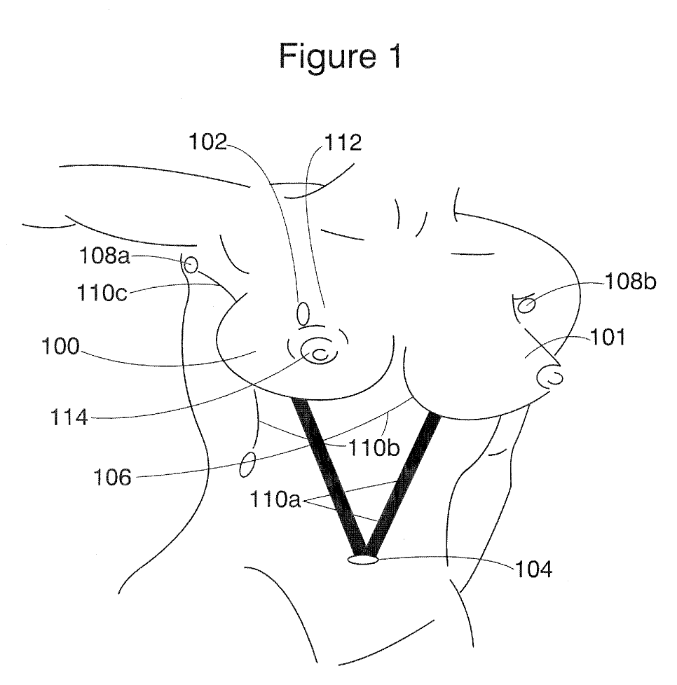

FIG. 1 illustrates one or more paths marked on the body of a patient that extends from an incision point to a pre-determined space behind the breasts, in accordance with various embodiments of the present invention. An incision is made at an incision point on the body of the patient. Examples of the incision point may include, but are not limited to, a point 102 on the breast 100, a naval 104, a pre-existing scar 106 and a pit point, such as a pit point 108a or a pit point 108b, under the arm pits of the patient.

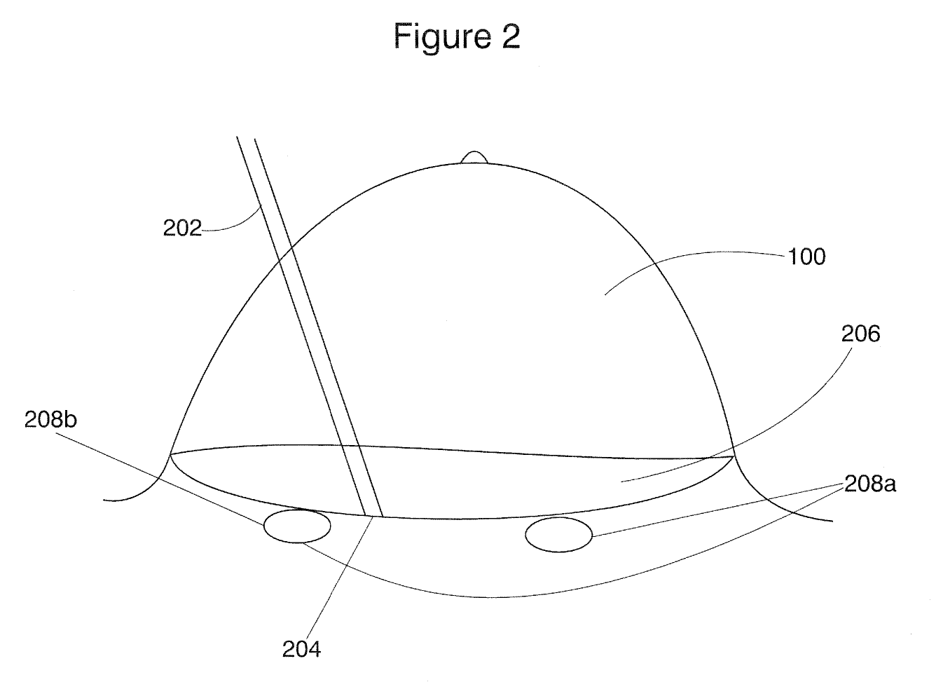

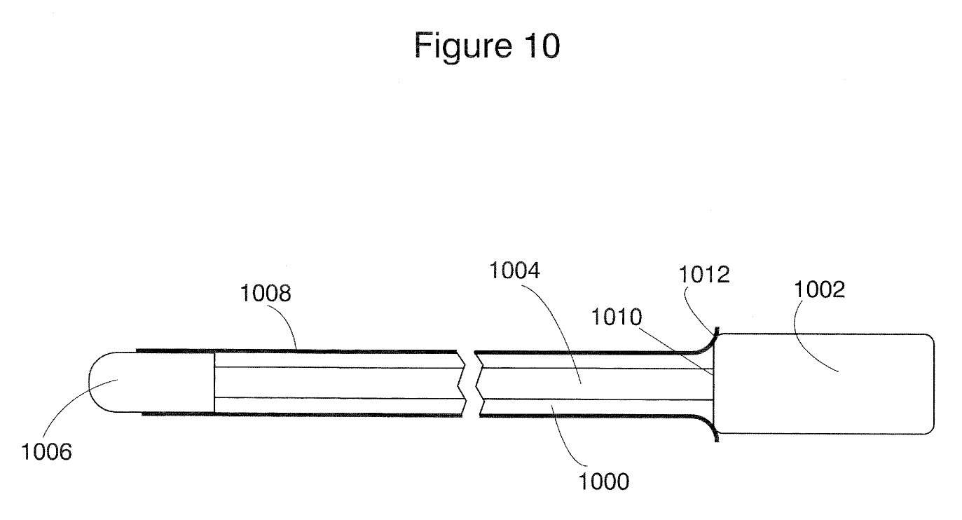

In an embodiment of the present invention, an implant is inserted percutaneously from the point 102 into the breast 100 of the patient. Technique of performing breast augmentation according to this embodiment of the present invention is explained in conjunction with FIGS. 2-9. In various embodiments of the present invention, the implant may be inserted percutaneously from the naval 104 or the pit points 108a, 108b into at least one of the breast 100 or 101 of the patient. The detail pertaining to breast augmentation through the naval 104 has been explained in detail in conjunction with FIGS. 10-22.

In various embodiments of the present invention, a path is marked on the skin of the patient for the guidance of the surgeon in to carry out the breast augmentation. For example, a path 110a is marked on the skin of the patient from the naval 104 to a pre-determined space 112 behind the breast 100. Similarly, a path 110b and a path 110c may be marked from the pre-existing scar 106 and a pit point 108a respectively, to the pre-determined space 112 behind the breast 100 of the patient. In an embodiment of the present invention, the pre-determined space 112 is medial to a nipple 114 and is about 6 to 8 centimeters above the level of the nipple 114.

It is to be understood that the technique of performing breast augmentation described with reference to the breast 100 is for the convenience of the reader and the technique is also applicable to the other breast 101 of the patient.

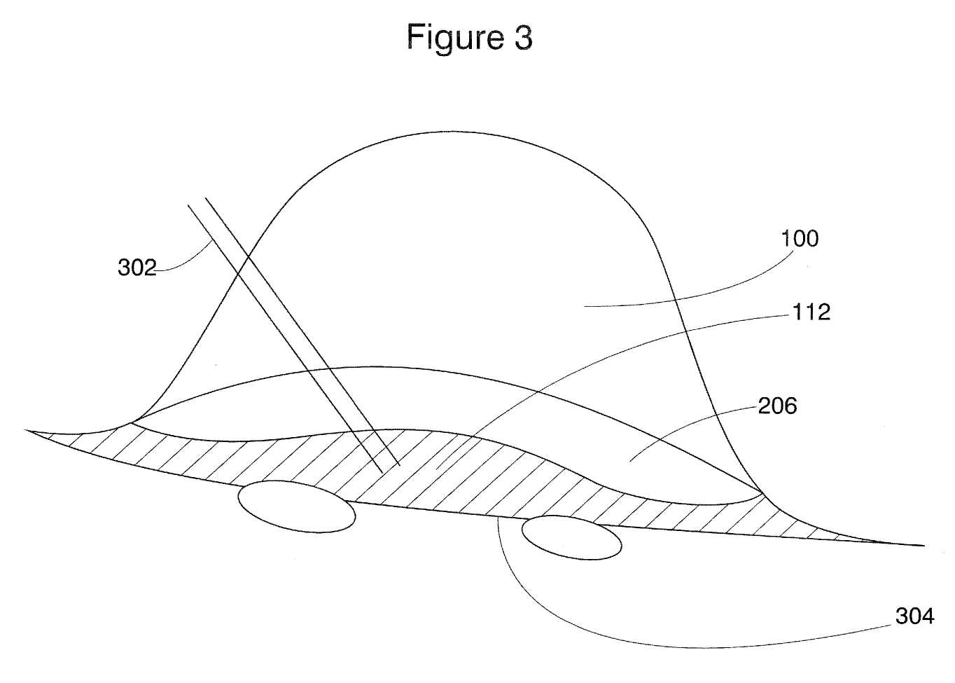

FIG. 2 illustrates a needle inserted into the breast of a patient, in accordance with an embodiment of the present invention. A needle 202, such as a seldinger needle, having a sharp stylet is inserted from the point 102 (not shown in the figure) into the breast 100 of the patient. The needle 202 is inserted in such a manner that a tip 204 of the needle 202 is posterior to the pectoral muscle 206 but above ribs, such as ribs 208a and ribs 208b, of the patient. The needle 202 is inserted to create a path for the introduction of a cannula (not shown in the figure). In an embodiment of the present invention, the cannula may be inserted into the breast 100 along the needle 202. Details pertaining to the cannula are explained in conjunction with FIG. 3.

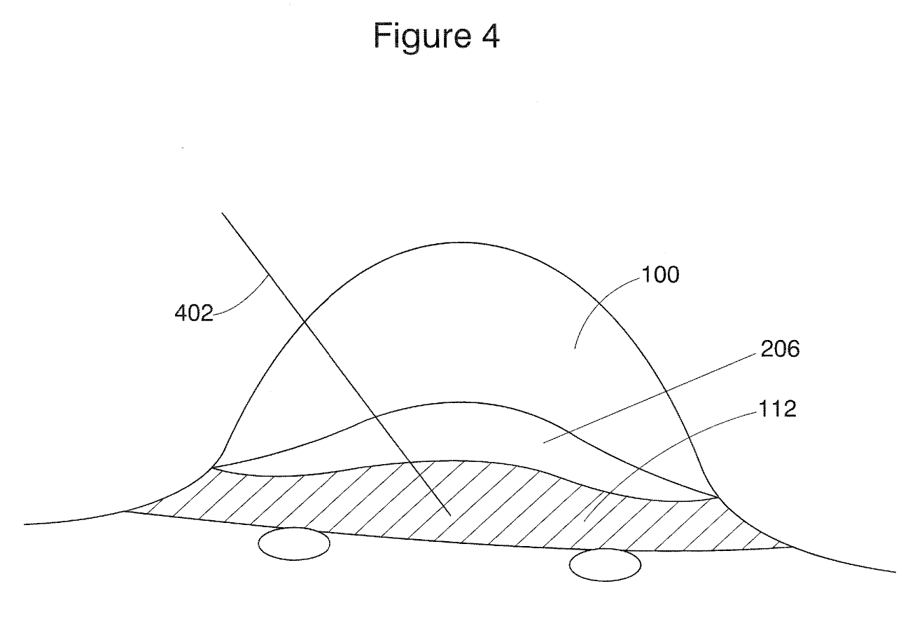

FIG. 3 illustrates a cannula inserted into the breast of the patient, in accordance with an embodiment of the present invention. A cannula 302 is inserted into the breast 100 over the needle 202 (not shown in the figure). The needle 202 is withdrawn from the cannula 302. Thereafter, a guide wire (not shown in the figure) is inserted through the cannula 302 into the breast 100 of the patient. The details pertaining to the guide wire are explained in conjunction with FIG. 4.

In an embodiment of the present invention, a fluid may be introduced through the cannula 302 to enlarge the pre-determined space 112 between the pectoral muscle 206 and a chest wall 304. An example of the fluid may include, but is not limited to, an anesthetic, such as lidocaine.

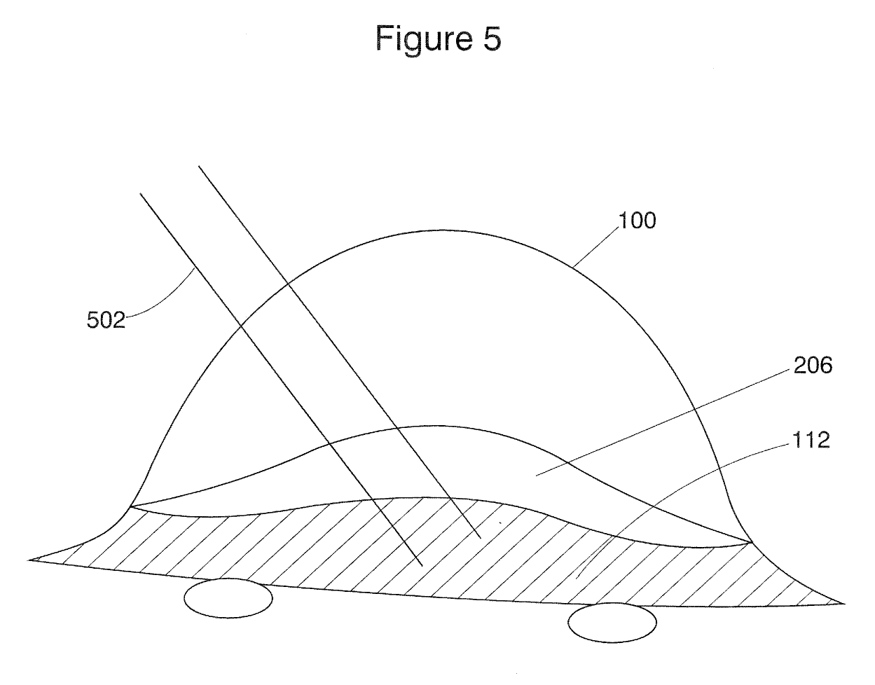

FIG. 4 illustrates a guide wire inserted through the cannula into the breast of the patient, in accordance with an embodiment of the present invention. A guide wire 402 is inserted through the cannula 302 (not shown in the figure) to maintain an access track to the pre-determined space 112. In an embodiment of the present invention, the size of the access track around the guide wire 402 can be increased using a dilator or a series of dilators using well-known techniques. Guide wire 402 enables insertion of a sheath (not shown in the figure) to the pre-determined space 112, posterior to the pectoral muscle 206. Details pertaining to the insertion of the sheath are explained in detail in conjunction with FIG. 5. The cannula 302 is withdrawn after the insertion of the guide wire 402.

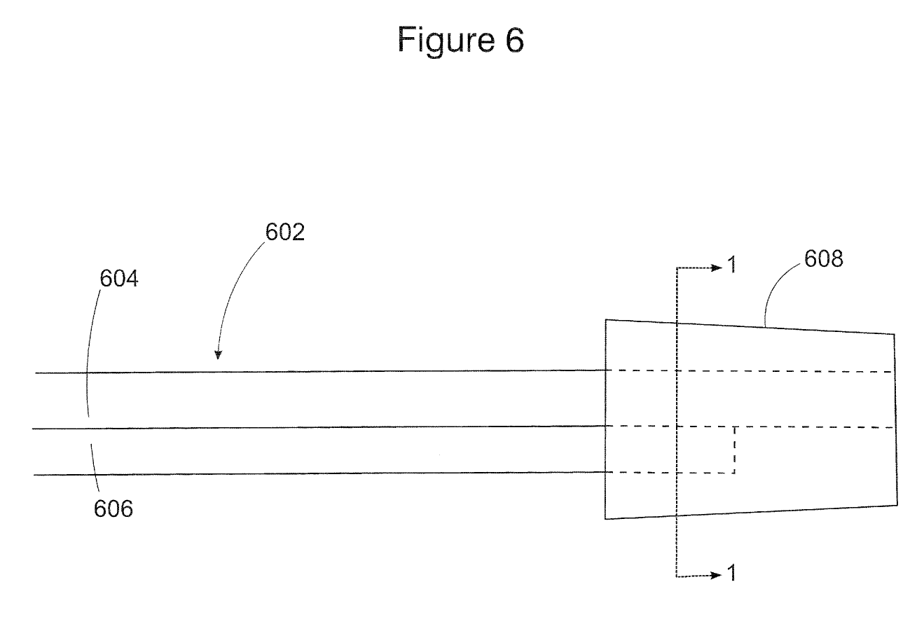

FIG. 5 illustrates a sheath inserted over the guide wire into the breast of the patient, in accordance with an embodiment of the present invention. A sheath 502 is inserted through the guide wire 402 from the access track to the pre-determined space 112, posterior to the pectoral muscle 206. The sheath 502 is introduced to maintain an access track for insertion of a catheter (not shown in the figure) to perform breast modification. Details pertaining to the catheter are explained in conjunction with FIGS. 6, 7 and 8.

In various embodiments of the present invention, the catheter may be inserted over the guide wire 402 without using the sheath 502.

FIG. 6 illustrates a detailed view of a catheter with an implant, in accordance with an embodiment of the present invention. A catheter 602 includes a guiding tube 604 and a feeding tube 606. The guiding tube 604 is fed over the guide wire 402 to guide the movement of the catheter 602 to the pre-determined space 112 using image guidance. Various image guidance techniques may be used to guide the movement of the tools, such as the needle 202, the cannula 302, the guide wire 402, the sheath 502 and the catheter 602 into the body of the patient.

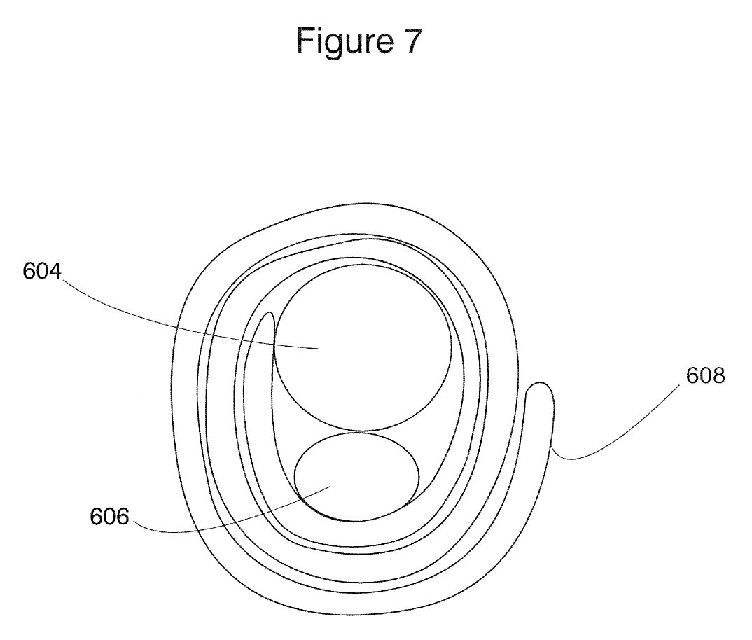

In an embodiment of the present invention, the catheter 602 may be attached with an implant 608, such as an inflatable balloon. Catheter 602 is divided along section lines 1-1 and its corresponding sectional view have been described in FIG. 7. A feeding tube 606 enables the insertion of fluids to inflate the implant 608.

In various embodiments of the present invention, the implant 608 may be connected to a sub-cutaneous inflation port to inflate the implant 608 to a pre-determined size. Further, the sub-cutaneous inflation port may adjust the size of the implant 608. In an embodiment of the present invention, the use of the sub-cutaneous inflation port for inflation may avoid the need of the feeding tube 606.

FIG. 7 illustrates a sectional view taken along the section lines 1-1 of the catheter with the implant, in accordance with an embodiment of the present invention. Implant 608 is attached with the catheter 602 through the feeding tube 606. The implant 608 is coupled to the feeding tube 606 by a self-sealing valve. In case the feeding tube 606 is withdrawn from the implant 608 the self-sealing valve seals the implant 608. In an embodiment of the present invention, the self-sealing valves are made from elastomeric materials that are well known in the art. Moreover, the self-sealing valves are described in U.S. Pat. No. 4,819,637 and U.S. Pat. No. 6,736,793 and the entire contents of these patents have been incorporated herein by reference. The implant 608 is wrapped around the catheter 602 to enable the insertion of the implant 608 through the sheath 502 into the breast 100 of the patient.

In an embodiment of the present invention, the feeding tube 606 is threaded into the self-sealing valve of the implant 608. The threading enables the feeding tube 606 to be securely coupled to the implant 608 while the tip of the feeding tube 606 extends through the self-sealing valve to be in position for inflating the implant 608.

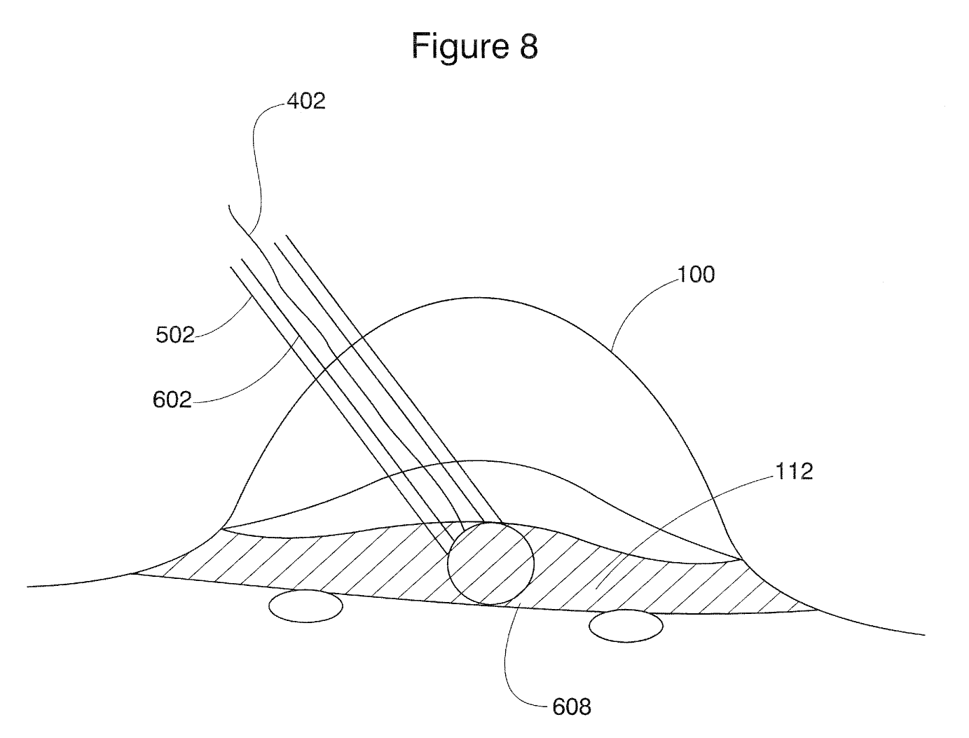

FIG. 8 illustrates the catheter inserted over the guide wire through the sheath into the breast of the patient, in accordance with an embodiment of the present invention. The catheter 602 with the implant 608 (as described in FIG. 6) is inserted over the guide wire 502, through the sheath 502, to the pre-determined space 112. Thereafter, a fluid, such as a saline solution, is inserted through the feeding tube 606 to inflate the implant 608 to a predetermined size. In various embodiments of the present invention, one or more fluids may be inserted to inflate the implant 608. These fluids are non-toxic and are non-reactive with the fluids in the body of the patient. In an embodiment of the present invention, the catheter 602 is rotated to enable unfurling of the implant 608 as the fluid is inserted into the implant 608.

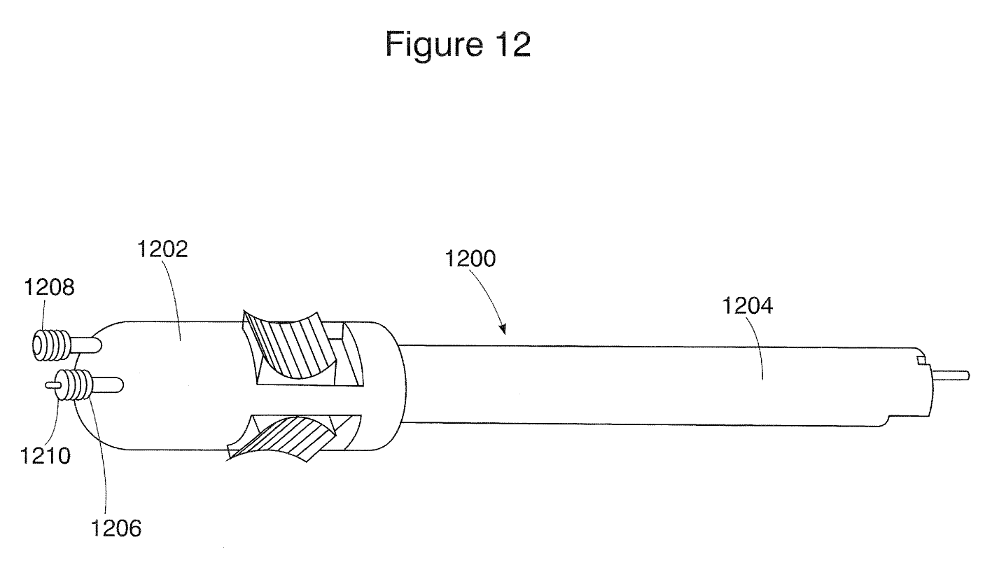

In various embodiments of the present invention, insertion of the needle 202, the cannula 302, the guide wire 402, the sheath 502 and the catheter 602 may lead to bleeding in the breast 100. In case bleeding is encountered, the pre-determined space 112 is irrigated by an irrigation instrument and suctioned by a suction instrument. Details pertaining to irrigation and suction instruments are explained in conjunction with FIGS. 12, 13, 14, 15, 16A, 16B, 17A and 17B.

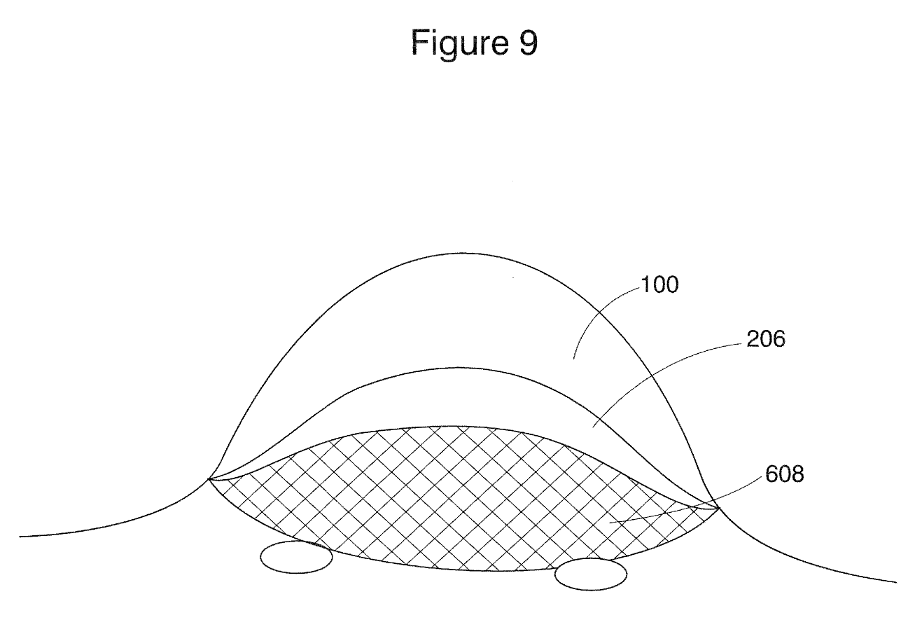

FIG. 9 illustrates the breast of a patient after augmentation, in accordance with an embodiment of the present invention. Once the implant 608 is sufficiently large, the implant 608 will be detached and the catheter 602 will be withdrawn. The point 102 in the breast 100 will then be closed.

In various embodiments of the present invention, the breast augmentation technique described above may be applied to other the breast 101 of the patient. Further, the same technique may be applied to buttock augmentation by inserting the implant 608 sub-gluteally into the body of the patient. Again this can be performed using ultrasound guidance and a percutaneous or endoscopic approach.

In various embodiments of the present invention, the pre-determined space 112 between the pectoral muscle 206 and the chest wall 304 may be enlarged using one or more inflatable balloons. Further, a hydrogel may be inserted into the pre-determined space 112 which may avoid the need of an inflated balloon.

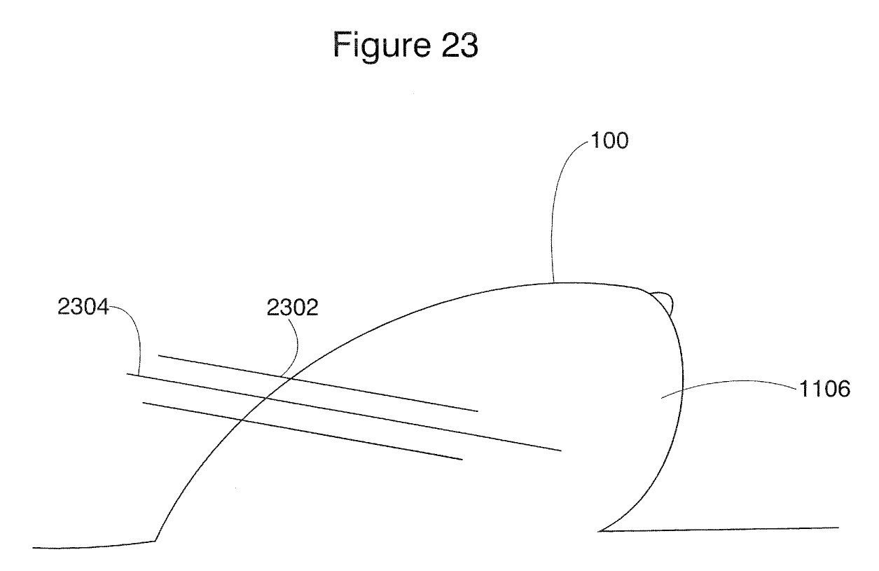

In an embodiment of the present invention, lifting of the breast 100 may be performed after the augmentation. Details related to the lifting of the breast 100 are explained in conjunction with FIGS. 23-27.

In another embodiment of the present invention, an implant may be inserted percutaneously under the pectoral muscle from the navel. Technique of performing breast augmentation according to this embodiment of the present invention is explained in conjunction with FIGS. 10-27. Note that a sub-pectoral implant placement using the naval endoscopic approach may be aided by endoscopic ultrasound guidance.

FIG. 10 illustrates a partial view of an endotube and an obturator for inserting into the body of the patient, in accordance with an embodiment of the present invention. An obturator 1000 includes an obturator handle 1002, an obturator rod 1004 and a bullet-shaped nose piece 1006. An endotube 1008 is a hollow tube with two ends, a flared end 1010 and a first distal end 1012. The obturator 1000 protrudes from the first distal end 1012 of the endotube 1008. The flared end 1010 of the endotube 1008 is adjacent to the obturator handle 1002. The bullet shaped nose piece 1006 is fixed partially inside the first distal end 1012.

In various embodiments of the present invention, the obturator 1000 and the endotube 1008 are made up of materials, such as surgical grade stainless steel or plastic of satisfactory strength. The surgical steel is preferred for instruments having long life. However, it may be desirable to make the instruments of a sterile plastic which may be disposed of after each operation. The diameter of the endotube 1008 and the obturator 1000 are based on the size of the surgical instruments which are to be introduced through the endotube 1008. For example, the obturator 1000 is about 24″ long and the endotube 1008 is about 16″ long, having an I.D. of 0.70″ and O.D. of 0.73″. The length of the endotube 1008 is based on the distance between the navel and the pre-determined space behind the breast of the patient. In an embodiment of the present invention, the distance from the flared end 1010 to the end of bullet-shaped nose piece 1006 is about 18″.

It is to be understood that the specific dimensions for the obturator 1000 and the endotube 1008 is for the convenience of the reader and is not to be construed as limiting these instruments to specific sizes.

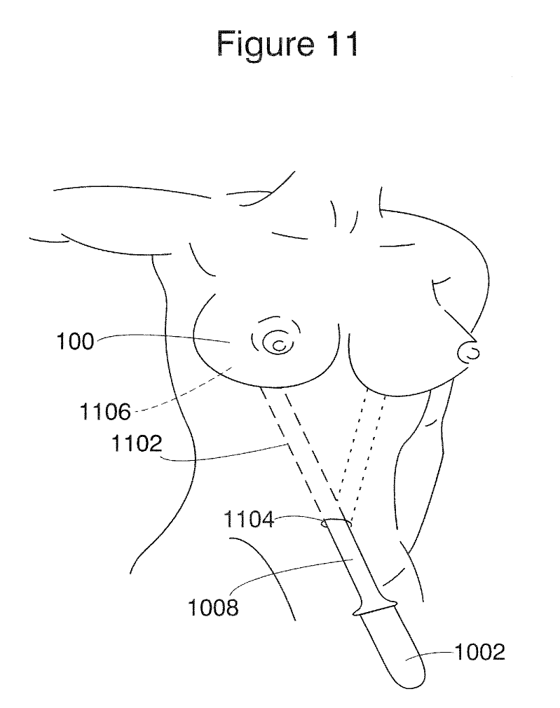

The endotube 1008 is inserted from the naval into the body of the patient. Details pertaining to the insertion of the endotube 1008 have been explained in conjunction with FIG. 11.

FIG. 11 illustrates a schematic view of the endotube that is partially inserted in the body of the patient, in accordance with an embodiment of the present invention. The endotube 1008, with the obturator 1000, is introduced into the body of the patient to form a tunnel 1102. The tunnel 1102 is formed from the naval 1104 to the pre-determined space 1106.

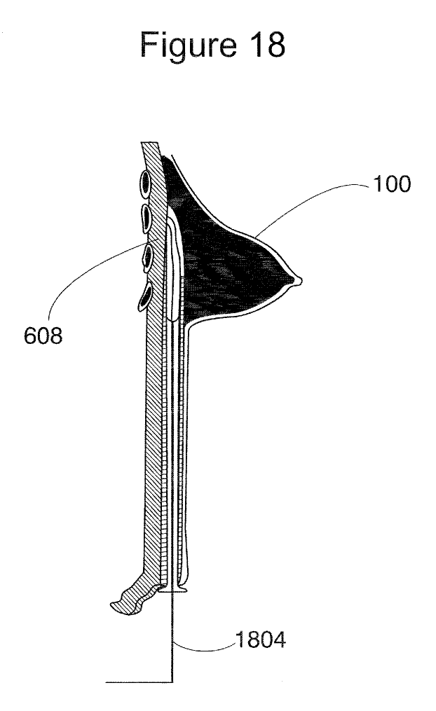

In one embodiment of the present invention, the endotube 1008 is passed to the pre-determined space 1106 from the umbilicus, staying just above the fascia of the interior abdominal and chest wall, and below the subcutaneous tissue and fat. The endotube 1008 is pushed over the rib cage from the naval 1104 leading to the pre-determined space 1106. During the insertion of the endotube 1008, the endotube 1008 and the breast 100 are manipulated manually for proper positioning of the endotube 1008. Manual manipulation of the endotube 1008 and the breast 100 has been explained in detail in conjunction with FIG. 18.

In an embodiment of the present invention, an endoscope is introduced through the tunnel 1102 to verify the position of the tunnel 1102 and to inspect the pre-determined space 1106 in case of bleeding. When bleeding is encountered, the pre-determined space 1106 is cleaned by irrigating and suctioning of the pre-determined space 1106 by inserting a combined instrument into the endotube 1008. Irrigating and suctioning of the pre-determined space 1106 by the combined instrument has been explained in detail in conjunction with FIGS. 12, 13, 14, 15, 16A, 16B, 17A and 17B.

In an embodiment of the present invention, an image guidance technique, such as ultrasound imaging, may be used to visualize the position of the organs and tools used in the breast augmentation. The ultrasound imaging helps the surgeon to adjust the tools, such as the combined instrument to follow the correct path to reach the pre-determined space 1106.

After the desired manipulation of the endotube 1008 and the breast 100, the obturator 1000 and the endotube 1008 are removed. Thereafter, an inflatable implant in inserted into the body of the patient through the endotube 1008. In an embodiment of the present invention, the implant and a feeding tube are introduced into the body of the patient through the endotube 1008. The feeding tube and the implant have been explained in detail in conjunction with FIG. 18. In an alternate embodiment of the present invention, a catheter may be inserted into the endotube 1008 to introduce the implant 608, such as a balloon, into the body of the patient. In an embodiment of the present invention, the catheter 602 may be used to insert into the endotube 1008. Catheter 602 has been explained in detail in conjunction with FIGS. 6 and 7.

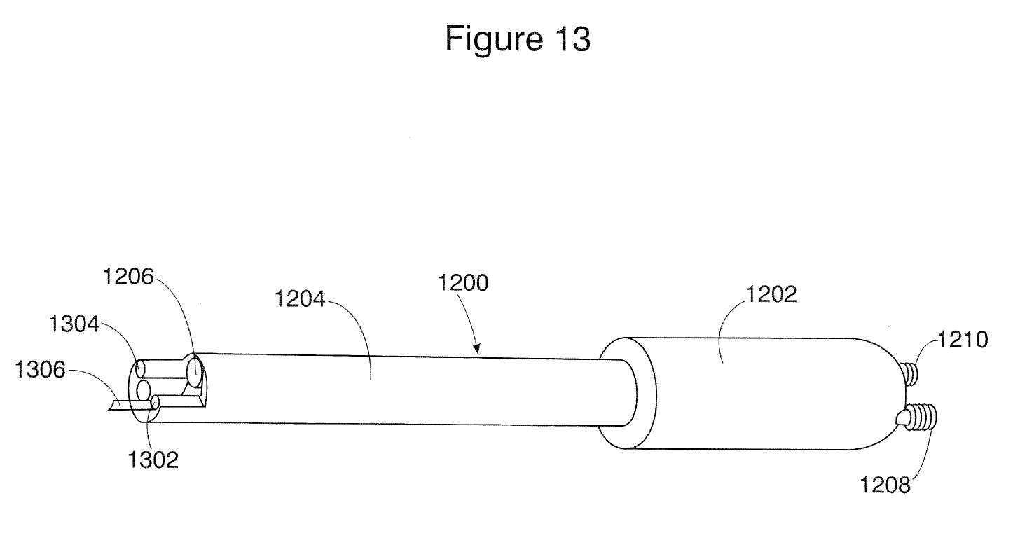

FIGS. 12 and 13 illustrate an isometric view from two different ends of the combined instrument, in accordance with an embodiment of the present invention. The combined instrument 1200 includes an instrument handle 1202, an instrument body portion 1204 and one or more passages. In case bleeding is encountered or cutting or cauterization is required, the combined instrument 1200 is introduced through the endotube 1008.

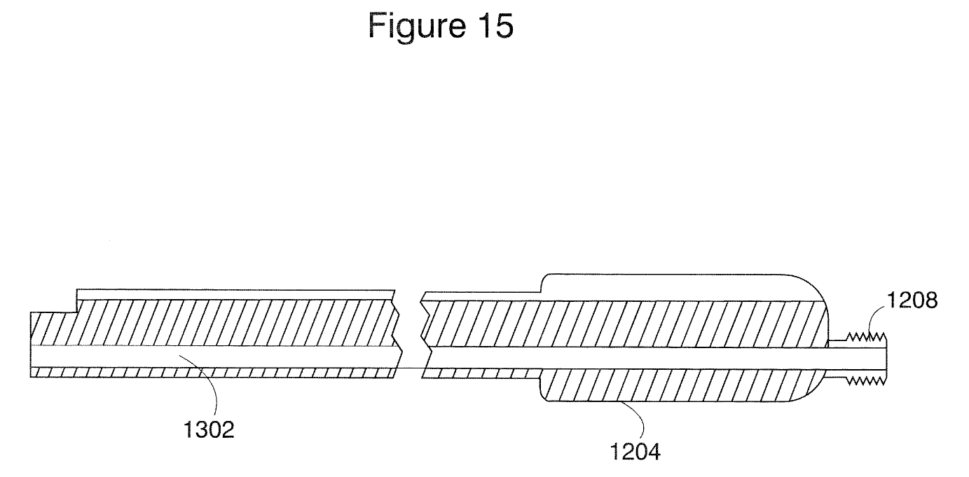

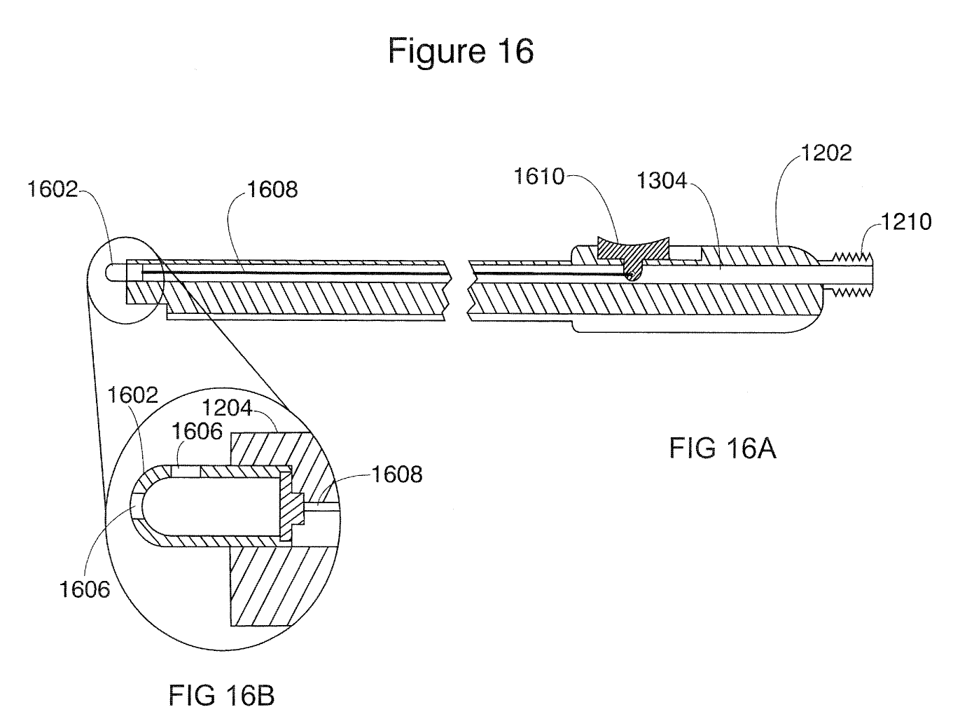

In one embodiment of the present invention, the one or more passages, such as a longitudinal passage 1206 may receive an endoscope to observe the pre-determined space 1106. In second embodiment of the present invention, the one or more passages, such as irrigation passage 1302 enables irrigation of the pre-determined space 1106. The combined instrument 1200 includes an irrigation inlet fitting 1208 for connecting to an irrigation tube (not shown in the figure). The irrigation passage 1302 has been explained in detail in conjunction with FIG. 15. In third embodiment of the present invention, the one or more passages, such as a suction passage 1304, to enable suctioning from the pre-determined space 1106. The combined instrument 1200 includes a sunction inlet fitting 1210 for connecting to a vacuum tube for suctioning the pre-determined space 1106. The suction passage 1304 has been explained in detail in conjunction with FIG. 16A and FIG. 16B.

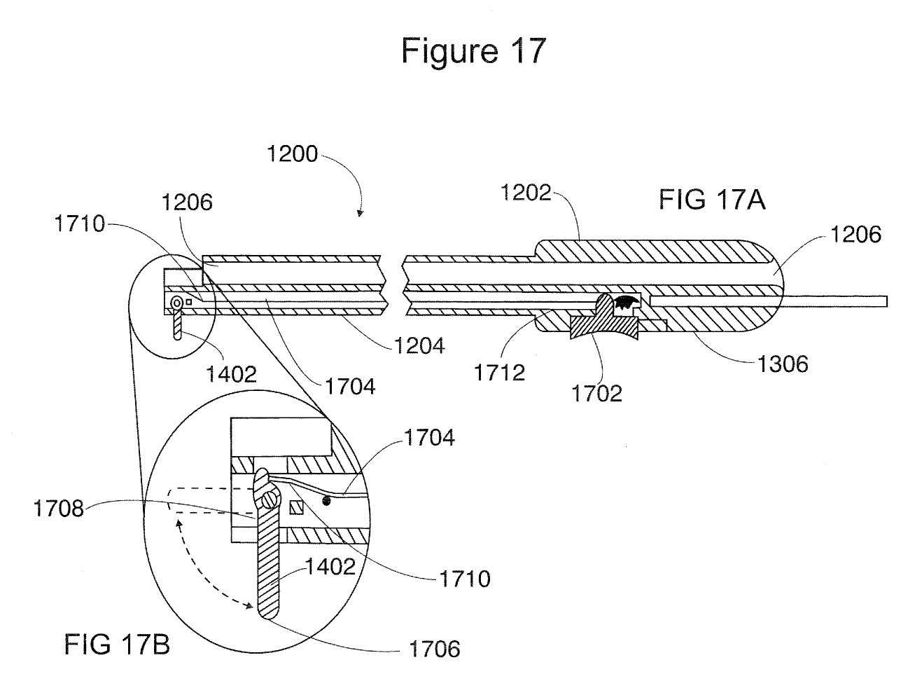

In fourth embodiment of the present invention, one or more cutting and cauterizing passages 1306 are used for electro cutting and cauterization of tissues. The cutting and cauterizing passage 1306 has been explained in detail in conjunction with FIG. 17A and FIG. 17B.

In various embodiments of the present invention, the one or more passages may be combined to form a single passage.

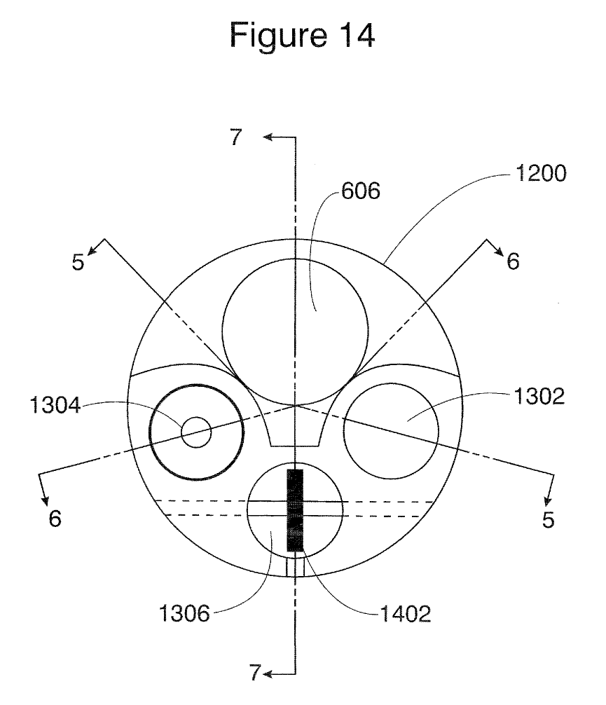

FIG. 14 illustrates a cross sectional view of the combined instrument, in accordance with an embodiment of the present invention. The combined instrument 1200 includes one or more passages, such as the longitudinal passage 1206, the irrigation passage 1302, the suction passage 1304 and the cutting and cauterizing passage 1306. The cutting and cauterizing passage 1306 includes a blade electrode 1402 for electrocuring or cauterization. Combined instrument 1200 is divided along section lines 5-5, section lines 6-6 and section lines 7-7 and its corresponding sectional views have been described in FIGS. 15, 16A, and 17A respectively.

FIG. 15 illustrates a sectional view taken on the section lines 5-5 in FIG. 14 of the combined instrument showing the irrigation passage, in accordance with an embodiment of the present invention. The irrigation passage 1302 includes the irrigation inlet fitting 1208 for connecting to an irrigation tube (not shown in figure). The pre-determined space 1106 is irrigated by the irrigation tube through the irrigation inlet fitting 1208.

FIG. 16A illustrates a sectional view taken on the section lines 6-6 in FIG. 14 of the combined instrument showing the suction passage, in accordance with an embodiment of the present invention. FIG. 16B illustrates a detailed view of the adjustable suction tip on the combined instrument, in accordance with an embodiment of the present invention. The suction passage 1304 includes the suction inlet fitting 1210 for connecting to a suction tube (not shown in figure). The suction passage 1304 also includes an extensible nozzle 1602 and an outlet end 1604. The extensible nozzle 1602 is positioned in an outlet end 1604 of the suction passage 1304 and is used to create a vacuum at the pre-determined space 1106. The extensible nozzle 1602 includes an aperture 1606, an operating rod 1608 and a nozzle lever 1610. The extensible nozzle 1602 provides a longitudinal extension and retraction by sliding over the aperture 1606. The aperture 1606 is movable by actuating the nozzle lever 1610. The nozzle lever 1610 is pivoted on the instrument handle 1202. The extensible nozzle 1602 creates the vacuum by suctioning the pre-determined space 1106 based on the movement of the nozzle lever 1610.

FIG. 17A illustrates a sectional view taken on the section lines 7-7 of the combined instrument showing the electro surgical, electro cauterizing and endoscopic viewing passage of the combined instrument, in accordance with an embodiment of the present invention. FIG. 17B illustrates a detail view of the electro surgical or electro cauterizing head of the combined instrument, in accordance with an embodiment of the present invention. The cutting and cauterizing passage 1306 is used for electro cutting and cauterization of tissues. The cutting and cauterizing passage 1306 includes the blade electrode 1402, a cutting lever 1702 and a blade linkage 1704. The blade electrode 1402 includes a blade tip 1706 and a pivot tip 1708. The blade linkage 1704 includes a linkage distal end 1712 along with a pivotal end 1710. The pivot tip 1708 is pivoted to the pivotal end 1710 of the blade linkage 1704. The cutting lever 1702 is connected to the blade electrode 1402 through the linkage distal end 1712 for extending or retracting the blade electrode 1402. Extension and retraction of the blade linkage 1704 lead to movement of the blade electrode 1402. Movement of the blade electrode 1402 permits cutting and cauterization of the tissues in the area defined by a circle having radius equal to the length of the blade electrode 1402.

In an embodiment of the present invention, the combined instrument 1200 includes a laser cutting and cauterizing element. In another embodiment of the present invention, the combined instrument 1200 includes an electro cautery endopod or a laser endopod. Each of the electro cautery endopod and the laser endopod includes a suction tube, an irrigation tube and a small caliber endoscope. The electro cautery endopod and the laser endopod further include a flexible or movable electro cautery tip and a fiber optic laser tip, respectively. The electro cautery tip and the fiber optic laser tip may be used for sharp dissection of the tissues.

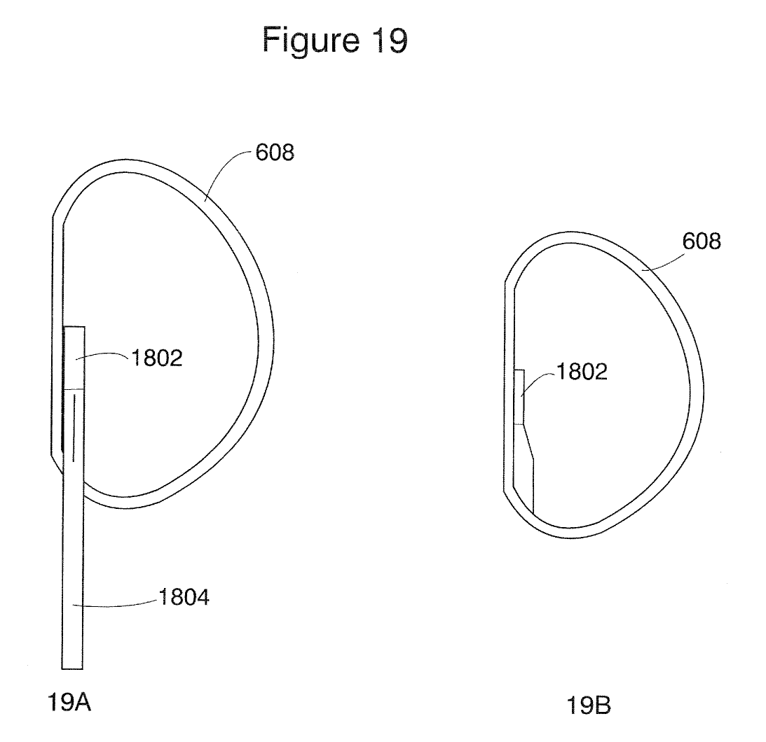

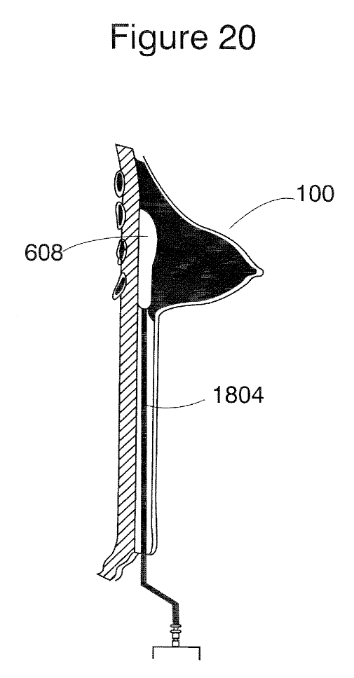

FIG. 18 illustrates the feeding tube and the implant inserted beneath the breast, in accordance with an embodiment of the present invention. An implant 608 has a self-sealing valve 1802 associated with it. The implant 608 is completely evacuated of air and rolled up tightly. The implant 608 is coupled to a feeding tube 1804 by the self-sealing valve 1802 (as shown in FIG. 19A and FIG. 19B). The feeding tube 1804 with the implant 608 is passed into the tunnel 1102 through the endotube 1008 to the pre-determined space 1106. Position of the implant 608 and the feeding tube 1804 is explained in conjunction with the FIG. 19A and 19B. The implant 608 is held manually by the operator by squeezing the breast 100. Simultaneously, the endotube 1008 is withdrawn to leave the implant 608 in place with the feeding tube 1804 coming out through tunnel 1102. The implant 608 is inflated by feeding a desired fluid through the feeding tube 1804. The feeding of the desired fluid has been explained in detail in conjunction with FIG. 20.

FIGS. 19A and 19B illustrate the position of the implant over the feeding tube, in accordance with an embodiment of the present invention. If the feeding tube 1804 on the implant 608 is considered to be in the 6 o'clock position, the implant 608 is positioned in such a way that the self-sealing valve 1802 is on the underside. From the 3 o'clock position the implant 608 is rolled up to the midline, then from the 9 o'clock position the other half of the implant 608 is rolled up to the midline. In an embodiment of the present invention, an FDA approved mammary implant having the desired structure may be used. Example of the implant 608 may include, but is not limited to, the RTV saline-filled mammary implant manufactured by Mc-Ghan Medical Corporation.

FIG. 20 illustrates filing of the implant through the feeding tube, in accordance with an embodiment of the present invention. A desired fluid, such as a saline solution, is passed through the feeding tube 1804 to inflate the implant 608. In an embodiment of the present invention, conventional fluids for breast implant may be used for inflating the implant 608. The implant 608 is overfilled to a volume that is greater than a pre-defined volume of the implant 608. In an embodiment of the present invention, the over-inflation volume is about 50% greater than the pre-determined volume. During the inflation of the implant 608, the breast 100 may be manually manipulated to shape and form a pocket in the pre-determined space 1106. In one embodiment of the present invention, in case the implant 608 is to be implanted in the sub-glandular or the sub-mammary position, the endotube 1008 and the breast 100 are manipulated in such a way that the endotube 1008 is kept below the breast tissue, but above the pectoralis muscle fascia. In second embodiment of the present invention, in case the implant 608 is to be implanted in the sub-muscular position, the endotube 1008 and the breast 100 are manipulated to allow penetration of the pectoralis muscle fibers and the endotube 1008 is directed to a sub muscular position. In an embodiment of the present invention, the breast 100 may be manipulated by a surgeon by holding and releasing pressure on the breast 100 in an appropriate manner. The over-inflation volume of the fluid is removed and the feeding tube 1804 is detached from the implant 608.

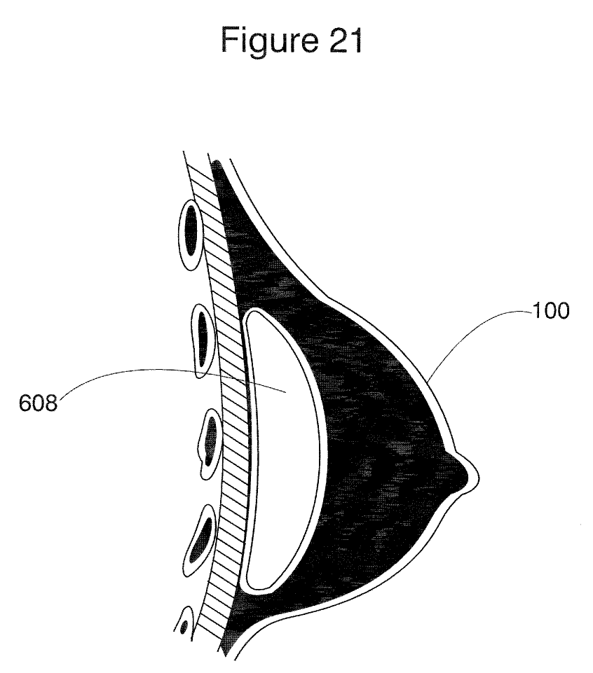

FIG. 21 illustrates a sectional view of the breast showing the implant in place. Thereafter, the endotube 1008 is withdrawn from the body of the patient. At the conclusion of the procedure, the incision point is then sutured.

In various embodiments of the present invention, both the breasts 100 and 101 are inspected by ultrasound imaging to ensure that there is no pooling of the fluid and there is no excessive bleeding.

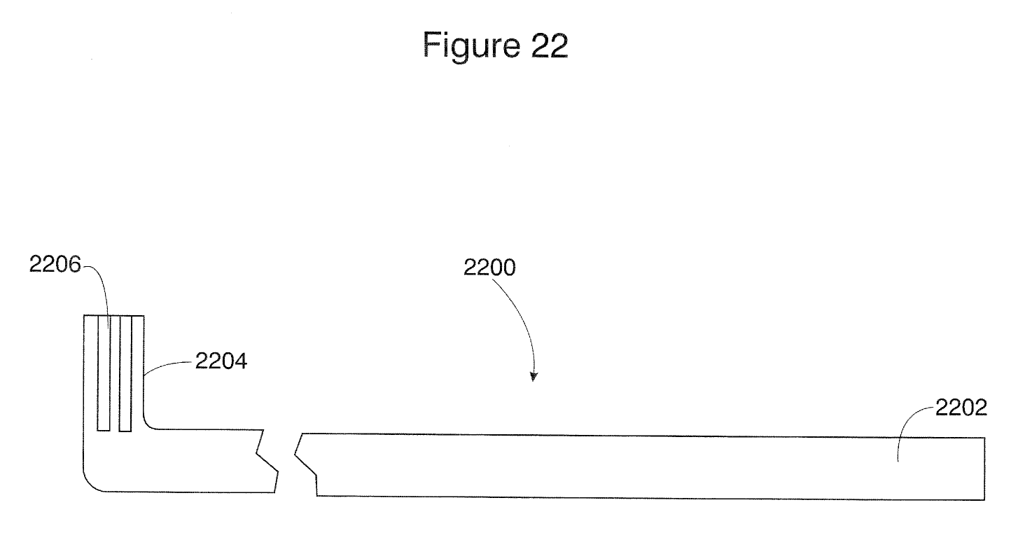

In an embodiment of the present invention, a blind-blunt dissection may be performed using a dissector, to enlarge the size of the pre-determined space 1106. In an embodiment of the present invention the blind-blunt dissection may be performed by a “hockey stick” dissector or a “Y” tipped dissector. The blind-blunt dissection has been explained in detail in conjunction with FIG. 22. Size of the pre-determined space 1106 may be enlarged by releasing muscular or fibrous bands that could not be released by expansion of the implant 608 or manipulations by the surgeon. Thereafter, the muscular or fibrous bands are aspirated from the pre-determined space 1106 by suctioning through the suction passage 1304.

FIG. 22 illustrates a plan view of a dissector used in separating tissues, in accordance with an embodiment of the present invention. The “hockey stick” dissector 2200 includes a dissector handle 2202 and a bent portion 2204. The bent portion 2204 includes a plurality of grooves 2206 with sharp edges to facilitate dissection during the surgical procedure. The “hockey stick” dissector 2200 may be used in the lower portion of the pre-determined space 1106. The “Y” tipped dissector (not shown in the figure) may be used in the upper part of the pre-determined space 1106.

According to another aspect of the present invention, breast augmentation may be followed by lifting of breast 100. The lifting of the breast 100 may be performed by grasping the tissue in the breast 100 with a barbed needle and anchoring the barbed needle to a rib with a suture. Details related to the lifting of the breast 100 are explained in conjunction with FIGS. 23-27.

FIG. 23 illustrates a tissue anchor needle inserted through a trocar needle into the breast, in accordance with an embodiment of the present invention. A trocar needle 2302 is inserted into the deep sub-cutaneous tissue of the breast 100. A tissue anchor needle 2304 is inserted through the trocar needle 2302. In an embodiment of the present invention, the tissue anchor needle 2304 includes a flexible shaft and a structure similar to the Semi-Flex RITA Medical RF probes.

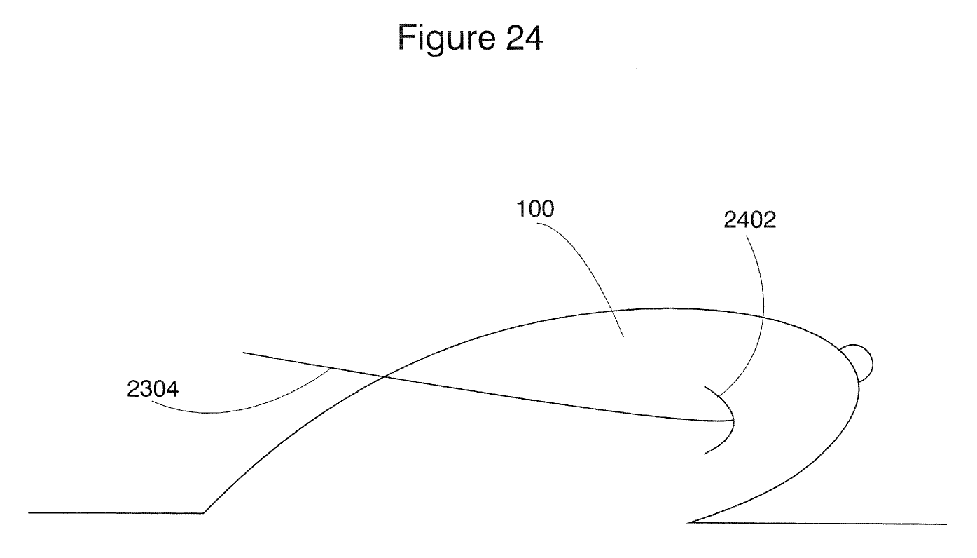

FIG. 24 illustrates deployment of barbs inside the breast by the tissue anchor needle, in accordance with an embodiment of the present invention. One or more barbs 2402 are deployed into the breast 100 from the end of the tissue anchor needle 2304. Thereafter, the trocar needle 2302 is withdrawn from the breast 100. In an embodiment of the present invention, the barbs 2402 may expand from the tissue anchor needle 2304 in all directions forming an umbrella-like array. In this embodiment, the opening through which the tissue anchor needle 2304 is inserted is dilated to enable insertion of barbs 2402. Barbs 2402 are inserted to hold the tissue and retain the tissue anchor needle 2304 into the breast 100 of the patient. In an embodiment of the present invention, barbs 2304 are designed to restrict the movement of the tissue anchor needle 2304 in a direction rearward to that of the insertion of the barb 2304. For example, design of barbs 2304 may be triangular in shape.

In an embodiment of the present invention, the tissue anchor needle 2304 is coated with material, such as Dacron mesh, that allows the tissue to grow onto the tissue anchor. In various embodiments of the present invention, the tips of the barbs 2402 are made of nitinol. The sharpness of the tips is such that the barbs 2402 should not cut through the tissue and the tissue is able to hold back the tissue anchor needle 2304.

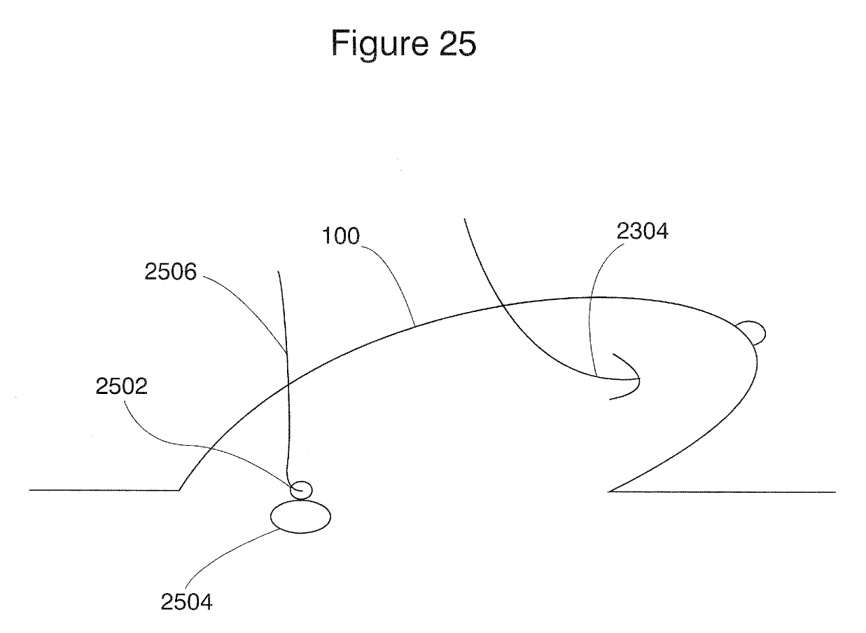

FIG. 25 illustrates a bone anchor introduced into the breast to the desired rib behind the breast, in accordance with an embodiment of the present invention. A bone anchor 2502 is connected to a rib 2504 after the tissue anchor needle 2304 is positioned into the sub-cutaneous tissue of the breast 100. The bone anchor 2502 is connected and introduced to the rib 2504 through a sheath such that the tip of the bone anchor 2502 is posterior to the pectoral muscle 206. In an embodiment of the present invention, the shape of the bone anchor 2502 is similar to an eye screw. In an embodiment of the present invention, a track is created to the rib 2504 to insert the sheath with the needle 202 and cannula 302. In another embodiment of the present invention, the track may be created with the trocar needle 2302. The track may be dilated and followed by insertion of the sheath to allow access to the rib 2504. Thereafter, the bone anchor 2502 is screwed into the rib 2504 with tools known in the prior art.

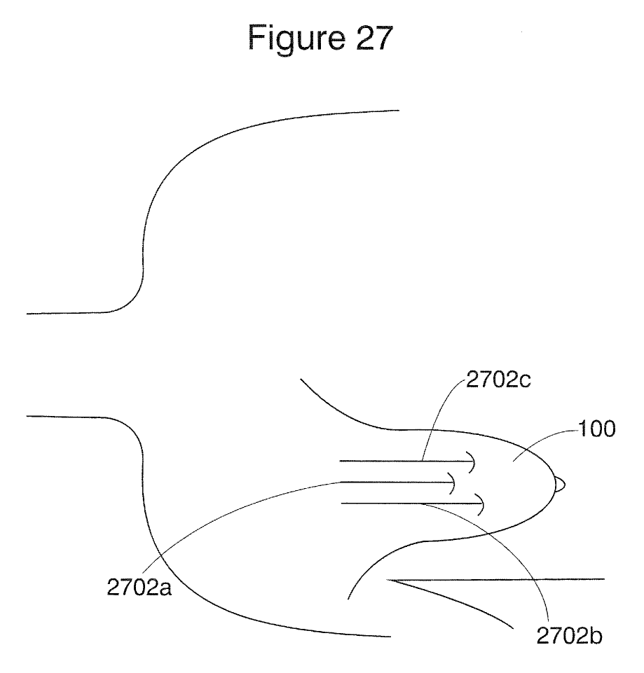

The suture 2506 is attached to the bone anchor 2502 and extends out of the breast 100. Suture 2506 connects the tissue anchor with the bone anchor 2502. In various embodiments of the present invention, one or more tissue anchors/bone anchors may be inserted into the breast 100. Insertion of the one or more tissue anchors/bone anchors are described in conjunction with FIG. 27.

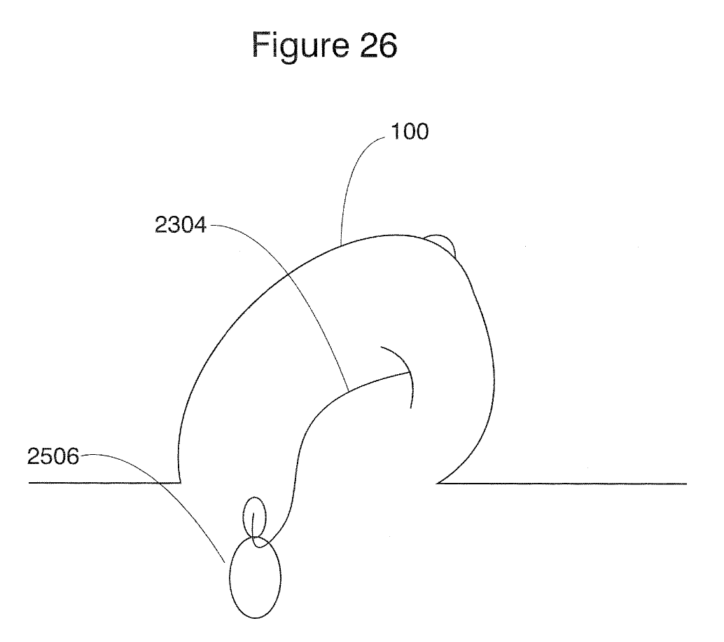

FIG. 26 illustrates adjustment of the bone anchor to desired amount to lift the breast, in accordance with an embodiment of the present invention. The tissue anchor needle 2304 is attached to the suture 2506. The breast 100 is lifted by adjusting the distance between the barbed end of the tissue anchor needle 2304 and the bone anchor 2502. Further, at the conclusion of the procedure, the incision point is sutured.

FIG. 27 illustrates multiple tissue anchor needles or multiple bone anchor needles inserted into the breast, in accordance with an embodiment of the present invention. One or more tissue anchor/bone anchor pairs, such as anchor pairs 2702a, 2702b and 2702c, may be inserted into the breast 100. Anchor pairs 2702a, 2702b and 2702c enables lifting of the breast 100.

In various embodiments of the present invention, the procedure described above for performing a breast lift may be used to perform a buttock lift, by securing the bone anchor to the pelvis.

Presently preferred embodiments of the invention have been described in detail hereinabove. However, various modifications and additions can be made without departing from the spirit and scope of the invention. In particular, the breast and buttock augmentations described above could conceivably be used to augment or lift other parts of the body without departing from the spirit and scope of the present invention. Accordingly, the foregoing description is meant to be taken only by way of example and not to otherwise limit the scope of the present invention as defined in the appended claims.

I claim:

1. An apparatus comprising:
a first tube having a lumen;
a second tube having a lumen that is coupled to the first tube to extend alongside the first tube;
an inflatable balloon including a valve that is detachably coupled to a distal end of the second tube such that a portion of the distal end of the second tube extends through the valve;
wherein the balloon is adapted to be introduced into a space in a body and inflated in the space in the body by fluid introduced through the valve through the second tube; and
wherein the second tube is detachable from the inflated balloon to leave the inflated balloon positioned in the space in the body.

2. The apparatus according to claim 1, wherein the space in the body is a sub-pectoral space in a human patient.

3. The apparatus according to claim 1, wherein the space in the body is a sub-gluteal space in a human patient.

4. The apparatus according to claim 1, wherein the valve is self-sealing.

5. The apparatus according to claim 1, wherein the second tube is screwed into the balloon at a position of the valve to secure the distal end of the second tube extending through the valve.

6. A method comprising:
percutaneously inserting a needle inside of a cannula into a patient such that a distal end of the needle reaches a predetermined space in the body;
removing the needle and introducing a guide wire through the cannula;
removing the cannula;
inserting a catheter over the guide wire, the catheter having a balloon at a distal end thereof that is inflatable via an inflation catheter extending along side the catheter;
inflating the balloon, and detaching the balloon from the catheter;
extracting the catheter to leave the inflated balloon positioned in the space in the body.

7. The apparatus according to claim 6, wherein the space in the body is a sub-pectoral space in a human patient.

8. The apparatus according to claim 6, wherein the space in the body is a sub-gluteal space in a human patient.

9. An apparatus for lifting a breast, comprising:
a bone anchor adapted to be screwed into a rib;
a tissue anchor including a plurality of barbs to be lodged in breast tissue; and
a suture to connect the tissue anchor to the bone anchor;
wherein a length of the suture is set to a desired length to lift the breast by a desired amount.
